(19) United States
(12) Patent Application Publication
(10) Pub. No.: US 2023/0038102 A1
(43) Pub. Date: Feb. 9, 2023

(54) REPLACEMENT HEART VALVE IMPLANT

(71) Applicant: BOSTON SCIENTIFIC SCIMED, INC., MAPLE GROVE, MN (US)

(72) Inventors: Michael G. Hayes, Galway (IE); Tara Hogan, Galway (IE); Andrea Goldbey, Galway (IE)

(73) Assignee: BOSTON SCIENTIFIC SCIMED, INC., MAPLE GROVE, MN (US)

(21) Appl. No.: 17/880,153

(22) Filed: Aug. 3, 2022

Related U.S. Application Data
(60) Provisional application No. 63/229,433, filed on Aug. 4, 2021.

(51) Int. Cl. A61F 2/24

(52) U.S. Cl. CPC A61F 2/24; A61F 2230/0002; A61F 2220/0075; A61F 2250/0069

(57) ABSTRACT

A replacement heart valve implant may include an expandable framework configured to shift from a collapsed configuration to an expanded configuration, the expandable framework having an inflow end and an outflow end, a plurality of valve leaflets secured to the expandable framework, and a plurality of pockets secured to the expandable framework in a plurality of circumferential pocket rows. The plurality of pockets extends radially outward from the expandable framework to capture retrograde fluid flow around an exterior of the expandable framework within the plurality of pockets.

### CROSS-REFERENCE TO RELATED APPLICATIONS

This application claims the benefit of priority of U.S. Provisional Application No. 63/229,433 filed Aug. 4, 2021, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve implant.

### BACKGROUND

A wide variety of intracorporeal medical devices have been developed for medical use, for example, intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

In one example, a replacement heart valve implant may comprise an expandable framework configured to shift from a collapsed configuration to an expanded configuration, the expandable framework having an inflow end and an outflow end, a plurality of valve leaflets secured to the expandable framework, and a plurality of pockets secured to the expandable framework in a plurality of circumferential pocket rows. The plurality of pockets may extend radially outward from the expandable framework to capture retrograde fluid flow around an exterior of the expandable framework within the plurality of pockets.

In addition or alternatively to any example described herein, the replacement heart valve implant may further comprise an inner skirt extending along an inner surface of the expandable framework.

In addition or alternatively to any example described herein, the inner skirt is fixedly attached to the expandable framework.

In addition or alternatively to any example described herein, the plurality of pockets is substantially impervious to fluid.

In addition or alternatively to any example described herein, the plurality of pockets is disposed upstream of the plurality of valve leaflets.

In addition or alternatively to any example described herein, each of the plurality of pockets includes a first end facing toward the inflow end of the expandable framework and a second end facing toward the outflow end of the expandable framework.

In addition or alternatively to any example described herein, the first end is fixed directly to the expandable framework.

In addition or alternatively to any example described herein, the second end includes a free edge extending between circumferentially adjacent frame struts of the expandable framework.

In addition or alternatively to any example described herein, the expandable framework includes a plurality of biasing arms configured to bias the plurality of pockets radially outward.

In addition or alternatively to any example described herein, a replacement heart valve implant may comprise an expandable framework configured to shift from a collapsed configuration to an expanded configuration, the expandable framework having an inflow end and an outflow end, a plurality of valve leaflets secured to the expandable framework, and a plurality of pockets secured to the expandable framework in a plurality of circumferential pocket rows. First pockets of the plurality of pockets disposed within a first circumferential pocket row of the plurality of circumferential pocket rows may be circumferentially offset from second pockets of the plurality of pockets within a second circumferential pocket row of the plurality of circumferential pocket rows. The plurality of pockets may extend radially outward from the expandable framework to capture retrograde fluid flow around an exterior of the expandable framework within the plurality of pockets.

In addition or alternatively to any example described herein, the plurality of pockets is generally triangular in shape when viewed normal to a central longitudinal axis of the expandable framework.

In addition or alternatively to any example described herein, the plurality of pockets is formed from a polymeric material.

In addition or alternatively to any example described herein, the expandable framework includes a plurality of frame struts defining a plurality of interstices between adjacent frame struts. The replacement heart valve further includes an inner skirt extending along an inner surface of the expandable framework. The inner skirt seals each of the plurality of interstices in the first circumferential pocket row of the plurality of circumferential pocket rows.

In addition or alternatively to any example described herein, each frame strut within the plurality of circumferential pocket rows has only one pocket of the plurality of pockets directly attached thereto.

In addition or alternatively to any example described herein, a replacement heart valve implant may comprise an expandable framework configured to shift from a collapsed configuration to an expanded configuration, the expandable framework having an inflow end and an outflow end, wherein the expandable framework includes a plurality of frame struts defining a plurality of interstices between adjacent frame struts, a plurality of valve leaflets secured to the expandable framework, and a plurality of pockets secured to the expandable framework in a plurality of circumferential pocket rows. Each pocket of the plurality of pockets within at least one of the plurality of circumferential pocket rows may circumferentially span more than one of the plurality of interstices.

In addition or alternatively to any example described herein, at least one frame strut of the plurality of frame struts within each of the plurality of circumferential pocket rows is free of direct attachment to the plurality of pockets.

In addition or alternatively to any example described herein, two circumferentially adjacent frame struts of the plurality of frame struts within each of the plurality of circumferential pocket rows are free of direct attachment to the plurality of pockets.

In addition or alternatively to any example described herein, first pockets of the plurality of pockets disposed within a first circumferential pocket row of the plurality of circumferential pocket rows are circumferentially offset from second pockets of the plurality of pockets within a second circumferential pocket row of the plurality of circumferential pocket rows.

In addition or alternatively to any example described herein, first pockets of the plurality of pockets disposed within the first circumferential pocket row of the plurality of circumferential pocket rows at least partially circumferentially overlap second pockets of the plurality of pockets within the second circumferential pocket row of the plurality of circumferential pocket rows.

In addition or alternatively to any example described herein, at least a portion of each pocket of the plurality of pockets is fixed directly to an inner skirt extending along an inner surface of the expandable framework.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIGS. 1-2 illustrate selected aspects of a medical device system;

FIG. 3 illustrates selected aspects of a replacement heart valve implant associated with the medical device system of FIGS. 1-2;

FIG. 4 illustrates selected aspects of the replacement heart valve implant including a plurality of pockets;

FIG. 5 illustrates selected aspects of the replacement heart valve implant including a plurality of pockets; and

FIG. 6 illustrates selected aspects of the replacement heart valve implant including a plurality of biasing arms.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, every feature and/or element may not be shown in each drawing.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered the greatest possible dimension measured according to the intended usage, while a “minimum extent” may be considered the smallest possible dimension measured according to the intended usage. In some instances, an “extent” may generally be measured orthogonally within a plane and/or cross-section, but may be, as will be apparent from the particular context, measured differently — such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Traditionally, treatment of the cardiovascular system was often conducted by directly accessing the impacted part of the system. For example, treatment of a blockage in one or more of the coronary arteries was traditionally treated using coronary artery bypass surgery. As can be readily appreciated, such therapies are rather invasive to the patient and require significant recovery times and/or treatments. More recently, less invasive therapies have been developed, for example, where a blocked coronary artery could be accessed and treated via a percutaneous catheter (e.g., angioplasty). Such therapies have gained wide acceptance among patients and clinicians.

Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can have a serious effect on a human and could lead to a serious health condition and/or death if not dealt with properly. Treatment of defective heart valves poses other challenges in that the treatment often requires the repair or outright replacement of the defective heart valve. Such therapies may be highly invasive to the patient. Disclosed herein is an apparatus, system, and/or method that may be used for preparing and/or delivering a medical implant to a portion of the cardiovascular system in order to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the strut”, “the biasing arm”, or other features may be equally referred to all instances and quantities beyond one of said feature. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient's anatomy, such as but not limited to, arteries, veins, and/or other body lumens.

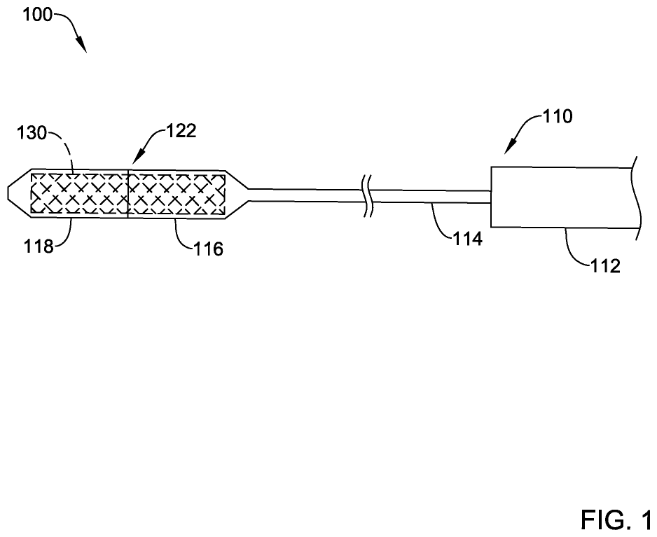

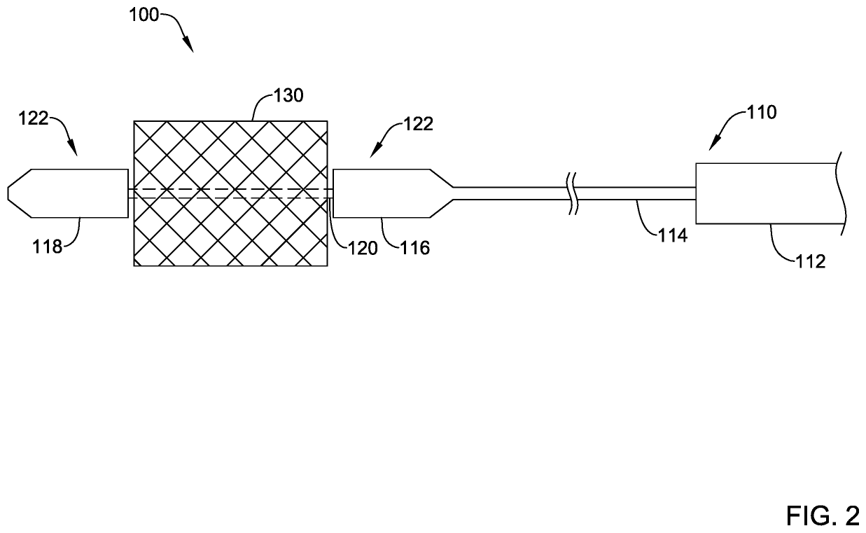

The medical device system 100, as seen in FIG. 1 for example, may generally be described as a catheter system that includes an implant delivery device 110 for delivering a replacement heart valve implant 130 which may be coupled to the implant delivery device 110 and disposed within a lumen of the implant delivery device 110 during delivery of the replacement heart valve implant 130. The implant delivery device 110 may include a proximal handle 112 and an elongate shaft 114 extending distally from the proximal handle 112. In some embodiments, the implant delivery device 110 and/or the elongate shaft 114 may include a proximal sheath 116 and a distal sheath 118. The implant delivery device 110 may include an inner shaft 120 (e.g., FIG. 2) slidably disposed within a lumen of the elongate shaft 114. The inner shaft 120 may be fixedly attached to the distal sheath 118. In some embodiments, the inner shaft 120 may include a guidewire lumen extending therethrough. In some embodiments, the proximal handle 112 may be configured to manipulate and/or translate the proximal sheath 116 and/or the distal sheath 118 relative to each other. In some embodiments, the proximal handle 112 may be configured to manipulate and/or translate the inner shaft 120 relative to the elongate shaft 114 and/or the proximal sheath 116.

During delivery of the replacement heart valve implant 130, the replacement heart valve implant 130 may be disposed within the proximal sheath 116 and/or the distal sheath 118 in a collapsed configuration, as seen in FIG. 1. In some embodiments, the proximal sheath 116 and/or the distal sheath 118 may collectively define a stent holding portion 122 of the implant delivery device 110. In some embodiments, the stent holding portion 122 may be configured to constrain the replacement heart valve implant 130 in the collapsed configuration. In some embodiments, the replacement heart valve implant 130 may be releasably coupled to the inner shaft 120.

In use, the medical device system 100 may be advanced percutaneously through the vasculature to a position adjacent to a treatment site. For example, the medical device system 100 may be advanced through the vasculature and across the aortic arch to a position adjacent to a defective aortic valve. Alternative approaches to treat a defective aortic valve and/or other heart valve(s) are also contemplated with the medical device system 100. After navigating the implant delivery device 110 and/or the stent holding portion 122 to the treatment site, the proximal sheath 116 and/or the distal sheath 118 may be translated relative to each other to open the stent holding portion 122. When unconstrained by the stent holding portion 122, the replacement heart valve implant 130 may be configured to shift from the collapsed configuration to an expanded configuration, as seen in FIG. 2. In at least some interventions, the replacement heart valve implant 130 may be deployed within the native valve (e.g., the native valve is left in place and not excised). Alternatively, the native valve may be removed (such as through valvuloplasty, for example) and the replacement heart valve implant 130 may be deployed in its place as a replacement. Some suitable but non-limiting materials for the medical device system 100, implant delivery device 110, the proximal handle 112, the elongate shaft 114, the proximal sheath 116, the distal sheath 118, the inner shaft 120, the stent holding portion 122, and/or components or elements thereof, for example metallic materials and/or polymeric materials, are described below.

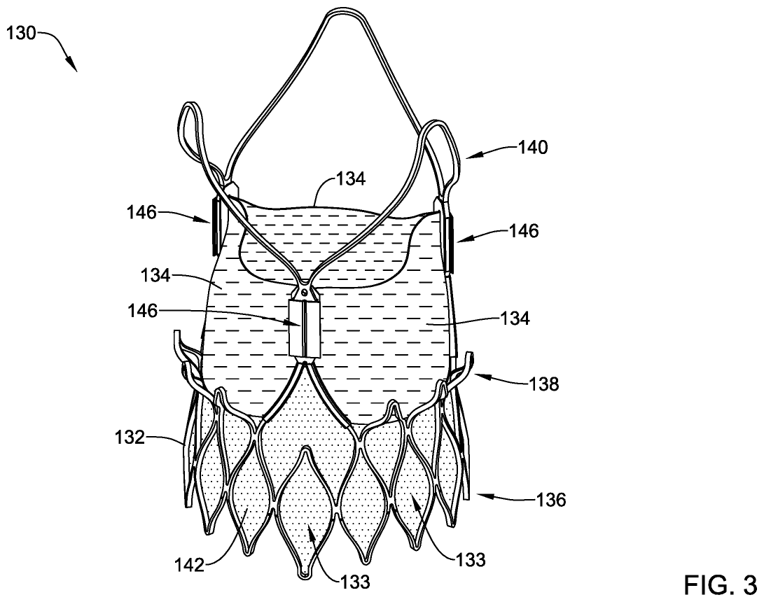

FIG. 3 illustrates selected aspects of a replacement heart valve implant 130. It should be appreciated that the replacement heart valve implant 130 can be any type of heart valve (e.g., a mitral valve, an aortic valve, etc.). In use, the replacement heart valve implant 130 can be implanted (e.g., surgically or through transcatheter delivery) in a mammalian heart. The replacement heart valve implant 130 can be configured to allow one-way flow through the replacement heart valve implant 130 from an inflow end to an outflow end.

The replacement heart valve implant 130 may include an expandable framework 132 defining a central lumen which, in some embodiments, may be substantially cylindrical. The side of the expandable framework 132 and other components facing the central lumen can be referred to as the luminal surface or luminal side. The opposite or outer side of the expandable framework 132 and other components (e.g., facing away from the central lumen) can be referred to as the abluminal surface or abluminal side. In some embodiments, the expandable framework 132 may have a substantially circular cross-section. In some embodiments, the expandable framework 132 can have a non-circular (e.g., D-shaped, elliptical, etc.) cross-section. In some embodiments, a non-circular expandable framework can be used to repair a mitral valve or another non-circular valve in the patient's heart or body. Some suitable but non-limiting examples of materials that may be used to form the expandable framework 132, including but not limited to metals and metal alloys, composites, ceramics, polymers, and the like, are described below.

The expandable framework 132 may be configured to shift from a collapsed configuration to an expanded configuration. In some embodiments, the expandable framework 132 may be self-expanding. In some embodiments, the expandable framework 132 may be self-biased toward the expanded configuration. In some embodiments, the expandable framework 132 may be mechanically expandable. In some embodiments, the expandable framework 132 may be balloon expandable. Other configurations are also contemplated. The expandable framework 132 may include a plurality of frame struts. The frame struts may define a framework or lattice structure. In some embodiments, the plurality of frame struts may define a plurality of interstices 133 (e.g., openings) between adjacent frame struts and/or through the expandable framework 132 from the luminal side to the abluminal side.

In some embodiments, the expandable framework 132 and/or the plurality of frame struts may define a lower crown 136, an upper crown 138, and a plurality of stabilization arches 140. In some embodiments, the lower crown 136 may be disposed at and/or may correspond to the inflow end of the expandable framework 132 and/or the replacement heart valve implant 130. In some embodiments, the upper crown 138 and/or the plurality of stabilization arches 140 may be disposed at and/or may correspond to the outflow end of the expandable framework 132 and/or the replacement heart valve implant 130. In some embodiments, the plurality of stabilization arches 140 may extend downstream of and/or away from the upper crown 138 in a direction opposite the lower crown 136. In some embodiments, the upper crown 138 may be disposed longitudinally and/or axially between the lower crown 136 and the plurality of stabilization arches 140.

The replacement heart valve implant 130 may include a plurality of valve leaflets 134 disposed within the central lumen. The plurality of valve leaflets 134 may be secured to the expandable framework 132. In some embodiments, the plurality of valve leaflets 134 may be fixedly attached to the expandable framework 132. Each of the plurality of valve leaflets 134 may include a root edge coupled to the expandable framework 132 and a free edge (e.g., a coaptation edge) movable relative to the root edge to coapt with the coaptation edges of the other leaflets along a coaptation region. In some embodiments, the plurality of valve leaflets 134 can be integrally formed with each other, such that the plurality of valve leaflets 134 is formed as a single unitary and/or monolithic unit. In some embodiments, a “root edge” can be a formed edge, such as when the plurality of valve leaflets 134 is formed in place on the expandable framework 132. In some embodiments, the plurality of valve leaflets 134 may be formed integrally with other structures such as an inner skirt 142 and/or an outer skirt (not shown), base structures, liners, or the like and in those circumstances the “root edge” is not a cut or otherwise divided edge, but rather is the location opposite the free edge where each of the plurality of valve leaflets 134 meets those other structures.

The free edges of the plurality of valve leaflets 134 may move into coaptation with one another in a closed position to substantially restrict fluid from flowing past the replacement heart valve implant 130. Specifically, the plurality of valve leaflets 134 may coapt to fill up or close the central lumen of the replacement heart valve implant 130 thereby impeding the flow of fluid in an upstream or retrograde direction. The free edges of the plurality of valve leaflets 134 may be move apart from each other in an open position to permit fluid flow through the replacement heart valve implant 130. Specifically, the plurality of valve leaflets 134 may move apart from each other to open the central lumen of the replacement heart valve implant 130 thereby permitting the flow of fluid in a downstream or antegrade direction. In FIG. 3, the plurality of valve leaflets 134 is shown in the open position or in a partially open position (e.g., a neutral position) that the plurality of valve leaflets 134 may move to when unbiased by fluid flow.

Each of the plurality of valve leaflets 134 may further include two connection portions. One connection portion can be disposed on either end of the free edge of its respective leaflet such that the connection portions are contacting or adjacent to the expandable framework 132 at a plurality of commissures 146. In some embodiments, the plurality of valve leaflets 134 may be secured and/or attached to the expandable framework 132 at the plurality of commissures 146. The free edges of the plurality of valve leaflets 134 may extend between the plurality of commissures 146.

In some embodiments, the plurality of commissures 146 may be disposed at a base of the plurality of stabilization arches 140. In some embodiments, each of the plurality of commissures 146 may join circumferentially adjacent stabilization arches of the plurality of stabilization arches 140 together. In some embodiments, the plurality of commissures 146 may be disposed longitudinally between the plurality of stabilization arches 140 and the upper crown 138. In some embodiments, the plurality of commissures 146 may be disposed distal of the plurality of stabilization arches 140 and proximal of the upper crown 138. In at least some embodiments, between circumferentially adjacent commissures of the plurality of commissures 146, the replacement heart valve implant 130 may be devoid of the expandable framework 132 at a longitudinal position radially outward of the free edges of the plurality of valve leaflets 134. As such, the free edges of the plurality of valve leaflets 134 may be free from direct contact with the expandable framework 132 as the plurality of valve leaflets 134 opens and/or closes.

In some embodiments, the connection portions of the plurality of valve leaflets 134 may also be referred to as commissural mounting tabs. In some embodiments, the connection portions may be disposed at least partially within a connection aperture defined and/or extending through the expandable framework 132 thereby coupling or attaching the plurality of valve leaflets 134 to the expandable framework 132. In some embodiments, the connection portions may be projections from their respective leaflet. In some embodiments, the connection portions may be integrally formed with its respective leaflet, such that the leaflet and connection portions are a single unitary and/or monolithic part or structure. In some embodiments, the connection portions of the leaflet can extend completely through the connection apertures, such as when the connection apertures extend completely through the expandable framework 132.

In some embodiments, the connection portions may encircle a portion of the expandable framework 132, such as when the connection portion contacts a strut at a location where the strut and/or the expandable framework 132 does not define a connection aperture. In some embodiments, the plurality of valve leaflets 134 and/or the connection portions may be attached to the expandable framework 132 using sutures, adhesives, or other suitable methods.

In some embodiments, the replacement heart valve implant 130 may include the inner skirt 142. In some embodiments, the inner skirt 142 may define a substantially tubular shape. The inner skirt 142 may be disposed on and/or extend along an inner surface (e.g., the luminal surface) of the expandable framework 132. In at least some embodiments, the inner skirt 142 may be fixedly attached to the expandable framework 132. The inner skirt 142 may direct fluid, such as blood, flowing through the replacement heart valve implant 130 toward the plurality of valve leaflets 134. In at least some embodiments, the inner skirt 142 may be fixedly attached to and/or integrally formed with the plurality of valve leaflets 134. The inner skirt 142 may ensure the fluid flows through the central lumen of the replacement heart valve implant 130 and does not flow around the plurality of valve leaflets 134 when they are in the closed position.

The inner skirt 142 may include a connection projection that extends from the inner skirt 142 and into one or more connection aperture. In some embodiments, the connection projection may extend around a portion of a strut and/or the expandable framework 132. In some embodiments, the connection projection may extend around a portion of a strut and into one or more connection aperture. In some embodiments, the connection projections may interact with the expandable framework 132 to attach or couple the inner skirt 142 to the expandable framework 132 through surface area contact and/or a form fitting configuration. In some embodiments, the connection projections may be attached to the expandable framework 132 using sutures, adhesives, or other suitable methods.

In some embodiments, the replacement heart valve implant 130 can include an outer skirt. In some embodiments, the outer skirt may define a substantially tubular shape. In some embodiments, the outer skirt may be disposed on the abluminal surface of the expandable framework 132. In some embodiments, the outer skirt may be disposed at and/or adjacent the lower crown 136. In some embodiments, the outer skirt may be disposed between the expandable framework 132 and the vessel wall in order to prevent fluid, such as blood, flowing around the replacement heart valve implant 130 and/or the expandable framework 132 in a downstream direction. The outer skirt may ensure the fluid flows through the replacement heart valve implant 130 and does not flow around the replacement heart valve implant 130, such as to ensure that the plurality of valve leaflets 134 can stop the flow of fluid when in the closed position.

The outer skirt may include a connection projection that extends from the outer skirt and into one or more connection aperture. In some embodiments, the connection projection may extend around a portion of a strut and/or the expandable framework 132. In some embodiments, the connection projection may extend around a portion of a strut and/or the expandable framework 132 and into one or more connection aperture. In some embodiments, the connection projections may interact with the expandable framework 132 to attach or couple the outer skirt to the expandable framework 132, such as through surface area contact or a form fitting configuration. In some embodiments, the connection projections may be attached to the expandable framework 132 using sutures, adhesives, or other suitable methods.

In some embodiments, the plurality of valve leaflets 134 may be comprised of a polymer, such as a thermoplastic polymer. In some embodiments, the plurality of valve leaflets 134 may include at least 50 percent by weight of a polymer. In some embodiments, the plurality of valve leaflets 134 may be formed from bovine pericardial or other living tissue. Other configurations and/or materials are also contemplated.

In some embodiments, the inner skirt 142 may include a polymer, such as a thermoplastic polymer. In some embodiments, the inner skirt 142 may include at least 50 percent by weight of a polymer. In some embodiments, the outer skirt may include a polymer, such as a thermoplastic polymer. In some embodiments, the outer skirt may include at least 50 percent by weight of a polymer. In some embodiments one or more of the plurality of valve leaflets 134, the inner skirt 142, and/or the outer skirt may be formed of the same polymer or polymers. In some embodiments, the polymer may be a polyurethane. Some suitable but non-limiting examples of materials that may be used to form the inner skirt 142 and/or the outer skirt, including but not limited to polymers, composites, and the like, are described below.

In some embodiments, the inner skirt 142 may be coupled to the lower crown 136 and/or the upper crown 138. In some embodiments, the inner skirt 142 may be coupled only to the upper crown 138. In some embodiments, the outer skirt may be coupled to the lower crown 136 and/or the upper crown 138. In some embodiments, the outer skirt may be coupled only to the lower crown 136. In some embodiments, the plurality of valve leaflets 134 may be coupled to the expandable framework 132 at a position that is at or just below the plurality of stabilization arches 140 and above the upper crown 138.

In some embodiments, the expandable framework 132 and/or the replacement heart valve implant 130 may have an outer extent of about 23 millimeters (mm), about 25 mm, about 27 mm, about 30 mm, etc. in an unconstrained configuration (e.g., in the expanded configuration). In some embodiments, the expandable framework 132 and/or the replacement heart valve implant 130 may have an outer extent of about 10 mm, about 9 mm about 8 mm, about 7 mm, about 6 mm, etc. in the collapsed configuration. Other configurations are also contemplated.

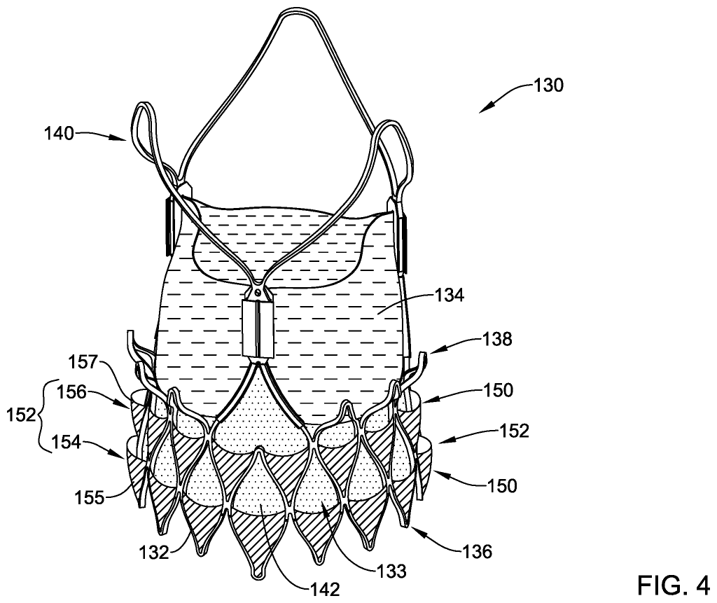

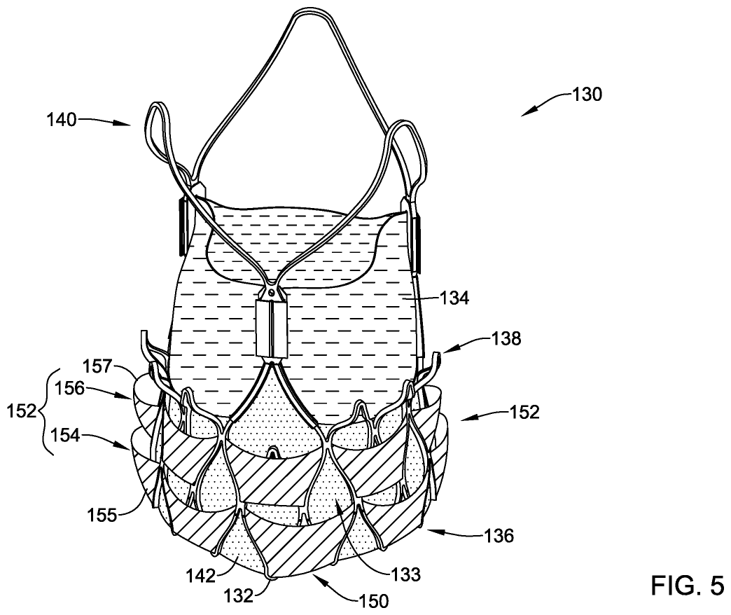

In some embodiments, the replacement heart valve implant 130 may include a plurality of pockets 150 secured to the expandable framework 132, as seen in FIGS. 4-5. In at least some embodiments, the plurality of pockets 150 may be fixedly attached to the expandable framework 132 and/or the inner skirt 142. In some embodiments, the plurality of pockets 150 may be disposed in a plurality of circumferential pocket rows 152 extending circumferentially about and/or around the expandable framework 132. In some embodiments, the plurality of pockets 150 may be disposed upstream of the plurality of valve leaflets 134 and/or the upper crown 138 of the expandable framework 132. For example, the plurality of pockets 150 may be disposed longitudinally closer to the lower crown 136 and/or the inflow end of the expandable framework 132 and/or the replacement heart valve implant 130 than the plurality of valve leaflets 134 is. In some embodiments, the plurality of pockets 150 may circumferentially span the plurality of interstices 133 formed in and/or defined by the expandable framework 132 and/or the plurality of frame struts.

In some embodiments, the plurality of pockets 150 may be substantially impervious to fluid. In some embodiments, the plurality of pockets 150 may be formed from a polymeric material. In some embodiments, the plurality of pockets 150 may be formed from a thin tissue (e.g., bovine pericardial, etc.). In some embodiments, the plurality of pockets 150 may be formed from a coated fabric material. In some embodiments, the plurality of pockets 150 may be formed from a nonporous and/or impermeable fabric material. Other configurations are also contemplated. Some suitable but non-limiting examples of materials that may be used to form the plurality of pockets 150 including but not limited to polymers, composites, and the like, are described below.

In some embodiments, the plurality of circumferential pocket rows 152 may include a first circumferential pocket row 154. In some embodiments, the plurality of circumferential pocket rows 152 may further include a second circumferential pocket row 156. In some embodiments, additional circumferential pocket rows may also be included. In some embodiments, first pockets 155 of the plurality of pockets 150 disposed within the first circumferential pocket row 154 of the plurality of circumferential pocket rows 152 may be circumferentially offset from second pockets 157 of the plurality of pockets 150 disposed within the second circumferential pocket row 156 of the plurality of circumferential pocket rows 152. In some embodiments, first pockets 155 of the plurality of pockets 150 disposed within the first circumferential pocket row 154 of the plurality of circumferential pocket rows 152 may at least partially circumferentially overlap second pockets 157 of the plurality of pockets 150 disposed within the second circumferential pocket row 156 of the plurality of circumferential pocket rows 152.

In some embodiments, the inner skirt 142 may seal each of the plurality of interstices 133 in the first circumferential pocket row 154 of the plurality of circumferential pocket rows 152. In some embodiments, the inner skirt 142 may seal each of the plurality of interstices 133 in the second circumferential pocket row 156 of the plurality of circumferential pocket rows 152. In some embodiments, the inner skirt 142 may seal each of the plurality of interstices 133 in the first circumferential pocket row 154 of the plurality of circumferential pocket rows 152 and in the second circumferential pocket row 156 of the plurality of circumferential pocket rows 152. In some embodiments, the inner skirt 142 may seal each of the plurality of interstices 133 formed in the expandable framework 132. In at least some embodiments, sealing the interstices as described herein may be considered to prevent fluid from flowing through the interstices from the luminal side of the expandable framework 132 to the abluminal side of the expandable framework 132. In some embodiments, the inner skirt 142 may be attached to the expandable framework 132 and/or the plurality of frame struts using one or more methods including but not limited to tying with sutures or filaments, adhesive bonding, melt bonding, embedding or over molding, welding, etc.

In some embodiments, each pocket of the plurality of pockets 150 may include a first end facing toward the lower crown 136 and/or the inflow end of the expandable framework 132 and/or the replacement heart valve implant 130. In some embodiments, the first end of each pocket of the plurality of pockets 150 may be fixed directly to the expandable framework 132 and/or the plurality of frame struts of the expandable framework 132. In some embodiments, each frame strut of the plurality of frame struts of the expandable framework 132 within the plurality of circumferential pocket rows 152 may have only one pocket of the plurality of pockets 150 directly attached thereto. In some embodiments, none of the plurality of pockets 150 is directly attached to, directly adjacent to, and/or adjoining another pocket of the plurality of the pockets 150. Other configurations are also contemplated. In some embodiments, each circumferential pocket row of the plurality of circumferential pocket rows 152 may be formed from a single unitary piece of material. In some embodiments, each pocket of the plurality of pockets 150 is formed from an independent and/or unique piece of material.

In some embodiments, each pocket of the plurality of pockets 150 may include a second end facing toward the upper crown 138 and/or the outflow end of the expandable framework 132 and/or the replacement heart valve implant 130. In some embodiments, the second end of each pocket of the plurality of pockets 150 may include a free edge extending between circumferentially adjacent frame struts of the plurality of frame struts and/or the expandable framework 132, as seen in FIG. 4. Accordingly, each pocket of the plurality of pockets 150 may circumferentially span one interstice of the plurality of interstices 133. In some embodiments, the plurality of pockets 150 may be generally triangular in shape when viewed normal to or along a radius extending from a central longitudinal axis of the expandable framework 132.

In some embodiments, the second end of each pocket of the plurality of pockets 150 may include a free edge extending between circumferentially spaced apart frame struts of the plurality of frame struts and/or the expandable framework 132, as seen in FIG. 5. In some embodiments, each pocket of the plurality of pockets 150 within at least one of the plurality of circumferential pocket rows 152 may circumferentially span more than one interstice of the plurality of interstices 133. In some embodiments, each pocket of the plurality of pockets 150 within at least one of the plurality of circumferential pocket rows 152 may circumferentially span two interstices of the plurality of interstices 133. In some embodiments, each pocket of the plurality of pockets 150 within each circumferential pocket row of the plurality of circumferential pocket rows 152 may circumferentially span more than one interstice of the plurality of interstices 133. In some embodiments, each pocket of the plurality of pockets 150 within each circumferential pocket row of the plurality of circumferential pocket rows 152 may circumferentially span two interstices of the plurality of interstices 133. Other configurations are also contemplated.

In some embodiments, at least one frame strut of the plurality of frame struts within each of the plurality of circumferential pocket rows 152 is free of direct attachment to the plurality of pockets 150. In some embodiments, two circumferentially adjacent frame struts of the plurality of frame struts within each of the plurality of circumferential pocket rows 152 are free of direct attachment to the plurality of pockets 150. In some embodiments, at least a portion of each pocket of the plurality of pockets 150 may be fixedly attached and/or may be fixed directly to the inner skirt 142 extending along and/or attached to the inner surface of the expandable framework 132. In some embodiments, the plurality of pockets 150 may be generally trapezoidal in shape when viewed normal to or along a radius extending from a central longitudinal axis of the expandable framework 132.

As seen in FIGS. 4 and 5, the plurality of pockets 150 may be configured to extend radially outward from the expandable framework 132 to capture retrograde fluid flow around an exterior of the expandable framework 132 within the plurality of pockets 150 to prevent fluid flow and/or aortic jetting through the native heart valve and back into the patient's heart (e.g., back into the left ventricle). In some embodiments, the plurality of pockets 150 may cooperate with the inner skirt 142 to capture retrograde fluid flow around the exterior of the expandable framework 132 within the plurality of pockets 150 to prevent fluid flow and/or aortic jetting through the native heart valve and back into the patient's heart (e.g., back into the left ventricle). As such, the plurality of pockets 150 may be configured to prevent and/or substantially reduce paravalvular leakage around the expandable framework 132 during diastole.

In some embodiments, the plurality of pockets 150 may be formed from a highly flexible and/or compliant thin film material. In some embodiments, the plurality of pockets 150 may be configured to conform to the annulus of the native heart valve. In some embodiments, the plurality of pockets 150 may be configured to occupy space between the expandable framework 132 and the annulus of the native heart valve. In some embodiments, the plurality of pockets 150 may be configured to conform to stenosis, calcification, calcium nodules, etc. associated with the native heart valve and/or surrounding tissue(s). In some patients, stenosis, calcification, calcium nodules, etc. may cause the native heart valve to have or assume an irregular shape, such that deployment of a replacement heart valve implant may not obtain sufficient sealing around the expandable framework due to gaps and/or space left between the expandable framework and the annulus of the native heart valve and/or surrounding tissue(s). The plurality of pockets 150 of the replacement heart valve implant 130 of the current disclosure may be configured to fill those gaps and/or space(s) during diastole by preventing fluid or blood from passing around the replacement heart valve implant 130 and back into the patient's heart.

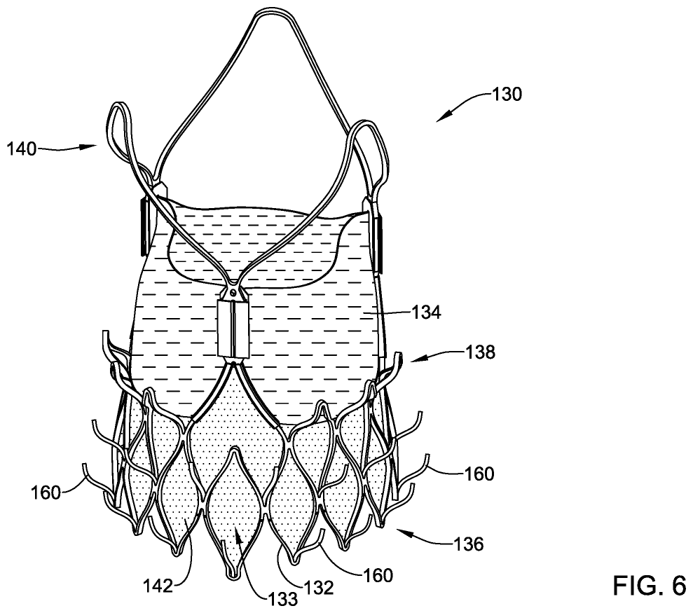

In some embodiments, the replacement heart valve implant 130 and/or the expandable framework 132 may include a plurality of biasing arms 160, as seen in FIG. 6. In FIG. 6, the plurality of pockets 150 is not shown for clarity. The plurality of biasing arms 160 may be configured to shift from a delivery configuration to a deployed configuration. In FIG. 6, the plurality of biasing arms 160 is shown in the deployed configuration.

In some embodiments, the plurality of biasing arms 160 may be generally aligned with the expandable framework 132 in the delivery configuration. In some embodiments, the plurality of biasing arms 160 may be oriented generally parallel to a central longitudinal axis of the expandable framework 132 in the delivery configuration. In some embodiments, the plurality of biasing arms 160 may be configured to extend radially outward from the expandable framework 132 in the deployed configuration. In some embodiments, the plurality of biasing arms 160 may be configured to extend radially outward from the expandable framework 132 in a downstream direction in the deployed configuration. In some embodiments, the plurality of biasing arms 160 may be configured to extend radially outward from the expandable framework 132 and longitudinally toward the outflow end of the replacement heart valve implant 130 and/or the expandable framework 132 in the deployed configuration.

In some embodiments, the plurality of biasing arms 160 may be heat set to a desired shape, angle, etc. in the deployed configuration. In some embodiments, the plurality of biasing arms 160 may be heat set at about 27 degree Celsius. In some embodiments, heat setting at 27 degrees Celsius may prevent the plurality of biasing arms 160 from protruding radially outward from the expandable framework 132 during assembly, sheathing, and/or delivery. In some embodiments, heat setting at 27 degrees Celsius may reduce sheathing forces. Other configurations are also contemplated. In some embodiments, the plurality of biasing arms 160 may include one or more curves formed therein in the deployed configuration. In some embodiments, the plurality of biasing arms 160 may be shaped similar to the upper crown 138 of the expandable framework 132 in the deployed configuration. Other configurations are also contemplated.

In some embodiments, the plurality of biasing arms 160 may be configured to bias the plurality of pockets 150 radially outward in the deployed configuration. In some embodiments, the plurality of biasing arms 160 may be directly attached to the expandable framework 132. In some embodiments, the plurality of biasing arms 160 may be integrally and/or monolithically formed with the expandable framework 132. In some embodiments, the plurality of biasing arms 160 may extend from intersections of adjacent frame struts of the plurality of frame struts of the expandable framework 132. In some embodiments, the plurality of biasing arms 160 may extend from the expandable framework 132 longitudinally toward the outflow end of the replacement heart valve implant 130 and/or the expandable framework 132 in the delivery configuration and/or the deployed configuration.

In some embodiments, the plurality of biasing arms 160 may extend into the plurality of interstices 133 in the delivery configuration. In some embodiments, one biasing arm of the plurality of biasing arms 160 may extend into at least one interstice of the plurality of interstices 133 in the delivery configuration. In some embodiments, one biasing arm of the plurality of biasing arms 160 may extend into each interstice of the plurality of interstices 133 in the delivery configuration. Other configurations are also contemplated. In some embodiments, areas and/or portions of the replacement heart valve implant 130 that may be located and/or placed in the vicinity of the membranous septum of the patient's heart may be devoid of the plurality of biasing arms 160 to reduce risk of pacing the patient's heart. Other configurations are also contemplated.

In at least some embodiments, the plurality of biasing arms 160 may be disposed radially outward of the inner skirt 142 in the delivery configuration and/or the deployed configuration. In some embodiments, the plurality of biasing arms 160 may engage and/or contact the plurality of pockets 150 in the deployed configuration. In some embodiments, the plurality of biasing arms 160 may prevent the plurality of pockets 150 from flexing, shifting, and/or moving radially inward toward the expandable framework 132.

In some embodiments, the plurality of biasing arms 160 may extend radially outward in the deployed configuration with enough radial force to urge and/or push the plurality of pockets 150 into voids, channels, gaps, and/or spaces formed between an exterior surface of the expandable framework 132 and the patient's anatomy (e.g., the annulus of the native heart valve and/or the surrounding tissue(s), etc.). In some embodiments, the plurality of biasing arms 160 may be configured to spring radially outward toward the deployed configuration upon implantation into the native heart valve. In some embodiments, the plurality of biasing arms 160 may aid and/or assist in anchoring the replacement heart valve implant 130 within the native heart valve.

The materials that can be used for the various components of the medical device system and the various elements thereof disclosed herein may include those commonly associated with medical devices. For simplicity purposes, the following discussion refers to the system. However, this is not intended to limit the devices and methods described herein, as the discussion may be applied to other elements, members, components, or devices disclosed herein, such as, but not limited to, the expandable framework, the inner skirt, the outer skirt, the plurality of leaflets, the plurality of pockets, the plurality of biasing arms, and/or elements or components thereof.

In some embodiments, the system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A replacement heart valve implant, comprising:
an expandable framework configured to shift from a collapsed configuration to an expanded configuration, the expandable framework having an inflow end and an outflow end;
a plurality of valve leaflets secured to the expandable framework; and
a plurality of pockets secured to the expandable framework in a plurality of circumferential pocket rows;
wherein the plurality of pockets extends radially outward from the expandable framework to capture retrograde fluid flow around an exterior of the expandable framework within the plurality of pockets.

2. The replacement heart valve implant of claim 1, further comprising an inner skirt extending along an inner surface of the expandable framework.

3. The replacement heart valve implant of claim 2, wherein the inner skirt is fixedly attached to the expandable framework.

4. The replacement heart valve implant of claim 1, wherein the plurality of pockets is substantially impervious to fluid.

5. The replacement heart valve implant of claim 1, wherein the plurality of pockets is disposed upstream of the plurality of valve leaflets.

6. The replacement heart valve implant of claim 1, wherein each of the plurality of pockets includes a first end facing toward the inflow end of the expandable framework and a second end facing toward the outflow end of the expandable framework.

7. The replacement heart valve implant of claim 6, wherein the first end is fixed directly to the expandable framework.

8. The replacement heart valve implant of claim 6, wherein the second end includes a free edge extending between circumferentially adjacent frame struts of the expandable framework.

9. The replacement heart valve implant of claim 1, wherein the expandable framework includes a plurality of biasing arms configured to bias the plurality of pockets radially outward.

10. A replacement heart valve implant, comprising:
an expandable framework configured to shift from a collapsed configuration to an expanded configuration, the expandable framework having an inflow end and an outflow end;
a plurality of valve leaflets secured to the expandable framework; and
a plurality of pockets secured to the expandable framework in a plurality of circumferential pocket rows;
wherein first pockets of the plurality of pockets disposed within a first circumferential pocket row of the plurality of circumferential pocket rows are circumferentially offset from second pockets of the plurality of pockets within a second circumferential pocket row of the plurality of circumferential pocket rows;
wherein the plurality of pockets extends radially outward from the expandable framework to capture retrograde fluid flow around an exterior of the expandable framework within the plurality of pockets.

11. The replacement heart valve implant of claim 10, wherein the plurality of pockets is generally triangular in shape when viewed normal to a central longitudinal axis of the expandable framework.

12. The replacement heart valve implant of claim 10, wherein the plurality of pockets is formed from a polymeric material.

13. The replacement heart valve implant of claim 10, wherein the expandable framework includes a plurality of frame struts defining a plurality of interstices between adjacent frame struts;
wherein the replacement heart valve further includes an inner skirt extending along an inner surface of the expandable framework;
wherein the inner skirt seals each of the plurality of interstices in the first circumferential pocket row of the plurality of circumferential pocket rows.

14. The replacement heart valve implant of claim 13, wherein each frame strut within the plurality of circumferential pocket rows has only one pocket of the plurality of pockets directly attached thereto.

15. A replacement heart valve implant, comprising:
an expandable framework configured to shift from a collapsed configuration to an expanded configuration, the expandable framework having an inflow end and an outflow end;
wherein the expandable framework includes a plurality of frame struts defining a plurality of interstices between adjacent frame struts;
a plurality of valve leaflets secured to the expandable framework; and
a plurality of pockets secured to the expandable framework in a plurality of circumferential pocket rows;
wherein each pocket of the plurality of pockets within at least one of the plurality of circumferential pocket rows circumferentially spans more than one of the plurality of interstices.

16. The replacement heart valve implant of claim 15, wherein at least one frame strut of the plurality of frame struts within each of the plurality of circumferential pocket rows is free of direct attachment to the plurality of pockets.

17. The replacement heart valve implant of claim 15, wherein two circumferentially adjacent frame struts of the plurality of frame struts within each of the plurality of circumferential pocket rows are free of direct attachment to the plurality of pockets.

18. The replacement heart valve implant of claim 15, wherein first pockets of the plurality of pockets disposed within a first circumferential pocket row of the plurality of circumferential pocket rows are circumferentially offset from second pockets of the plurality of pockets within a second circumferential pocket row of the plurality of circumferential pocket rows.

19. The replacement heart valve implant of claim 18, wherein first pockets of the plurality of pockets disposed within the first circumferential pocket row of the plurality of circumferential pocket rows at least partially circumferentially overlap second pockets of the plurality of pockets within the second circumferential pocket row of the plurality of circumferential pocket rows.

20. The replacement heart valve implant of claim 15, wherein at least a portion of each pocket of the plurality of pockets is fixed directly to an inner skirt extending along an inner surface of the expandable framework.
